(19) United States
(12) Patent Application Publication
(10) Pub. No.: US 2025/0380976 A1
(43) Pub. Date: Dec. 18, 2025

(54) APPARATUS AND METHODS FOR ORTHOPEDIC PROCEDURES SUCH AS TOTAL HIP REPLACEMENT AND BROACH AND STEM INSERTION

(71) Applicant: VALUE FORCES LTD., Haifa (IL)

(72) Inventors: Nir KARASIKOV, HAIFA (IL); James Randolph Dick, Muntinlupa City (PH); Nadav Cohen, Naifa (IL)

(21) Appl. No.: 18/879,627

(22) PCT Filed: Jul. 23, 2023
(86) PCT No.: PCT/IL2023/050767

§ 371 Date: Dec. 27, 2024

Related U.S. Application Data
(60) Provisional application No. 63/392,101, filed on Jul. 25, 2022.

(51) Int. Cl. A61B 17/92; A61B 17/00; A61B 34/00; A61B 34/30; A61F 2/46; G16H 20/40; G16H 40/63

(52) U.S. Cl. CPC A61B 17/92; A61B 34/30; A61B 34/76; A61F 2/4607; A61F 2/4609; G16H 20/40; G16H 40/63; A61B 2017/00115; A61F 2002/4632; A61F 2002/4666; A61F 2002/4668; A61F 2002/4681

(57) ABSTRACT

An apparatus and method for inserting a femoral broach or femoral stem or acetabular cup into a bone. The apparatus includes at least one closed loop insertion mechanism to provide impacts on the associated handles of the broach, stem, or the acetabular cup; at least one sensor to sense a series of impacts, including impact angle, and to provide real-time feedback of the alignment and/or condition of the bone; and a processor operably connected to the at least one sensor. The processor includes an algorithm that receives the real-time feedback to control the force, frequency, and alignment of the impacts by the at least one closed loop insertion mechanism.

### TECHNOLOGICAL FIELD

The presently disclosed subject matter is in the field of orthopedics, in particular, hip replacements and the like.

### BACKGROUND

A Total Hip Arthroplasty (THA) offers excellent results in patients suffering from end-stage osteoarthritis (OA). During total and partial hip replacement, a surgeon implants a femoral stem and an acetabular socket. There are two ways to fix the femoral stem to the femur bone, organic (cementless), which is by far the most common procedure, and cemented.

With cementless stem fixation, the surgeon inserts a series of broaches of gradually increasing size into the femoral canal, until getting the correct size broach, which is replaced by an artificial femoral stem upon which an acetabular prosthetic ball joint is fitted. The cementless stem fixation is based on a press-fit mechanism, the preparation of the medullary femoral canal is 1.5-2 mm smaller than the stem to be inserted.

To insert the broaches, the broaches are connected to a broach handle, which is repeatedly impacted with a surgical mallet. A series of broaches are used, starting from an initial small broach to successively larger broaches. If the broach is impacted with excessive force or in an off-axis direction, the femur bone could fracture or crack.

The impact strength is presently controlled subjectively, for example, by the sound emitted by a mallet/hammer blow on the broach or stem during insertion to the femur. There is no precise control of the impact intensity and direction and no feedback regarding the bone condition other than the impact noise and the physician's experience and intuition.

A major issue in hip replacements, in particular with total hip arthroplasty (THA) using cementless femoral stems, is the occurrence of intraoperative periprosthetic femoral fractures. Press-fit impaction is the most popular technique for the fixation of cementless femoral stems, which may lead to intraoperative periprosthetic femoral fractures. The incidence of intraoperative periprosthetic femoral fractures with cementless femoral stems during primary Total Hip Arthroplasty THA has been reported to be 3.5% to 5.4%. The incidence of periprosthetic femoral fractures during ‘revision’ THA is reported to be in the range of 13% to 21%. The incidence of ‘revision’ THA is reported to be 8% of all ‘primary’ THA procedures.

Broach alignment during impaction is an additional concern, which, if there is non-alignment, can contribute to bone fractures during the insertion of broaches and ultimately the insertion of the femoral stem. According to some reports, see https://pubmed.ncbi.nlm.nih.gov/28108173/, horizontally displaced forces (toward cortical bone) were magnified from 4% to a maximum value of 52%.” An off-axis impact direction can increase side forces by as much as 235%.

The acetabular cup insertion poses challenges to achieve sufficient insertion and orientation of the cup within the acetabulum to assure full motion range of the joint.

Related methods in the art include a jack hammer type device to replace the manual mallet, as illustrated in https://www.youtube.com/watch?v=o8LMFzQuuaE; and audible discernment by the surgeon of the change in the noise after each impact when the broach penetrates the hard cortical bone surface.

Publications relating to issues and experimentation regarding femoral stem insertion include:

PCT/IL2022/050646 (Karasikov, et al.), which discloses an apparatus and methods for total hip replacement broach and stem insertion.

Sakai, et al., “Hammering force during cementless total hip arthroplasty and risk microfracture”, Hip Int 2011; 21 (03) 330-335.

Krull, et al., “Maximizing the fixation strength of modular components by impaction without tissue damage”, Bone and Joint Research, vol. 7, No. 2, 196-204, 1 Feb. 2018.

Greenhill, et al. “Broach handle design changes for distribution in the femur during total hip arthroplasty”, The Journal of Arthroplasty 32 (2017) 2017-2022.

Tijou, et al., “Monitoring cementless femoral stem insertion by impact analyses: an in vitro study”, J Mech Behav Biomed Mater, 2018 December;88:102-108, doi: 10.1016/j.jmbbm.2018.08.009, Epub 2018 Aug. 10.

Tijou, et al., “Study Monitoring cementless femoral stem insertion by impact”, Journal of mechanical behavior of biomedical materials, Elsevier, 2018, https:/arxiv.org/abs/1905.08246.

Additional publications relevant to the technology include: U.S. Pat. No. 10,463,505 (Behzadi); U.S. Pat. No. 9,430,189 (Soles, et al.); US 2018/228614 (Lang, et al.); and WO 2021/174295 (Miles, et al.).

### GENERAL DESCRIPTION

The presently disclosed subject matter relates to a method and apparatus to help assure appropriate alignment of impacts and their force on a broach handle to insert a broach into a femoral bone so that the broach will progress and properly prepare the femoral canal for stem insertion, mitigating bone fracture. The subject matter also relates to the insertion of an acetabular cup into an acetabulum. A proper cementless stem insertion enables good bone ingrowth for a stable stem-to-bone fixation, and avoids fractures and loosening. Fractures or cracks can occur if the broach is too big or impacted too hard or at an incorrect angle. Loosening can develop if the stem is too small or insufficiently inserted in which case loosening of the stem occurs in the femur.

The presently disclosed subject matter provides an apparatus including a provision for alignment. The apparatus includes at least one sensor; at least one insertion mechanism; and a processor, with an alignment and force analysis algorithm (which preferably also analyzes output from the at least one sensor). The sensors may be any of (or combination of) a variety of sensors including accelerometer; ultrasonic; microphone; vibrometer; interferometer, load cell, position sensor; acoustic emission sensor; and transducer, which transmits and receives a vibration signal and acoustic radiation. In some examples, the at least one sensor is located between the broach or stem and the broach handle. The insertion mechanism is configured to impact on the broach/stem handle to mechanically insert the broach/stem into the bone in a controllable and quantifiable manner. In some examples, the alignment sensors are at least at one location on the perimeter of the broach handle, such as at four sides of the broach handle or connected to a contact plate sensor, and/or a sensor on the distal femur area. The insertion mechanism also ensures accurate insertion of the acetabular cup under sensor feedback for handheld devices or robotic platforms.

The processor/algorithm is configured to process output from the impact sensor(s) and/or the alignment sensors, to analyze impact direction and bone tension (stress), and to control the impaction of the insertion mechanism and its alignment to the bone, broach and broach handle, typically using feedback in real time from the sensors by analysis of shock waves (e.g. acoustic waves) produced during the impaction by the insertion mechanism on the handle; and by analysis of the shock wave variations detected by the sensors. The methodology is also applicable for acetabular cup insertion under acoustic feedback. The broach and stem impact alignment is conducted using controlled low-energy impacts to prevent bone damage. Once the alignment between the insertion mechanism, the broach handle, the broach and the bone, is set to assure optimal direction along the femur, the impaction with the required force is activated.

In case the impact sensor includes a transducer, a chirp wave could be generated during or right after the impact, and reflected waves are detected by the same, or another/auxiliary transducer. The reflected waves include information about the bone stress, e.g. through bone acoustic impedance measured by reflection and damping of the transducer waves-for example, by Fast Fourier Transform (FFT). The FFT shows the spectral response of the bone/stem structure, or acetabulum/acetabular cup structure, whereupon adequate fixation the FFT stabilizes the eigen frequencies of the bone/implant structures and does not change further with the insertion, and provides an input on sufficient insertion, to the impact analysis algorithm. The analysis may include comprehensive deep learning algorithms to learn the fingerprint of bone stress metrics when approaching critical stress, to assure optimal insertion via real time feedback about the bone (i.e. via the sensor) so when the broach, stem, or acetabular cup approaches the correct insertion position, the surgeon will receive an indication of the insertion status and the impact force is lowered by the system's analysis algorithm. Early prediction of a fracture will be indicated by the system at any point during the procedure to allow the surgeon to determine if surgery should continue or if the broach or stem sizes should be changed.

In some examples, the insertion mechanism is operably connected to the processor and controlled thereby. This control of the insertion mechanism by the processor, based on signals from the sensors, ensures proper force and direction and prevents use of excessive impact force. A subset of this feature is where the apparatus, via the insertion mechanism, provides impacting above a threshold impact force so that the broach will progress into the bone canal (i.e. initially above an elastic spring-back force and deflection of the soft tissue, and then of the cortex tissue), but within a range of impact force that ensures that the bone will not fracture.

In some examples of the apparatus for inserting the broaches or stem into the bone, the apparatus is used in association with a broach/stem handle and has sensors for measuring impaction; a closed loop insertion mechanism configured to provide a series of impacts on the handle; at least one impact sensor configured to sense the series of impacts, including the force and the vibration frequency of the impacts and their alignment, and to provide real-time feedback of a condition of the bone; at least one alignment sensor configured to determine the insertion mechanism alignment via assessment of directional stress; and a processor operably connected to those sensors and configured to receive the real-time feedback therefrom, and operably connected to the insertion mechanism to control the rate, angle and force, of the impacts that the insertion mechanism applies to the broach/stem handle.

In some examples, the alignment sensors, and the impact sensors, are any one or combination of an accelerometer, a vibrometer, an interferometer, a load cell, a position sensor, a wide band microphone, an acoustic emission sensor, and a transducer. By comparing the values of the plurality of sensors, the processor can determine the optimal impact direction/angle to provide a properly directed impact or to adjust the angle of the insertion mechanism. The direction can be determined also by a directional sensor that can determine the force direction.

The insertion mechanism can be held by a surgeon or by a robotic surgical platform. The apparatus allows impacting at relatively low force and provides either haptic or visual feedback to the surgeon (or analogous communication input with a robotic surgical platform) to indicate a proper impact direction, and if the impact angle needs to be adjusted. In the robotic case, the alignment sensors send feedback to the robot to adjust its angular degrees of motion and lateral impact position to assure alignment.

In some examples, the alignment sensors and the impact sensors are configured to provide real-time feedback based on shock-induced vibrational data or acoustic data.

In some examples, the processor includes an artificial intelligence system for multi-parameter data analysis.

In some examples, the insertion mechanism and/or a side alignment insertion mechanism is a piezoelectric insertion mechanism.

In some examples, the insertion mechanism and/or the side alignment insertion mechanism is an electro-magnetic insertion mechanism.

In some examples, the insertion mechanism is configured to provide impacts at a frequency of between 1 and 100 impacts per second.

In some examples, the insertion mechanism and/or the side alignment insertion mechanism includes a first excitable mass and a second hitting mass; a spring; and an excitation actuator.

In some examples, the excitation actuator is electromagnetic, either a motor; a solenoid; or a voice coil.

In some examples, the at least one impact sensor is mounted between the broach insertion handle and the broach, or between a stem insertion handle and the stem, or between an insertion handle and the acetabular cup,

In some examples, the at least one impact and/or alignment sensor is disposed on one or more sides of the insertion handle and/or the broach/stem.

In some examples, the at least one sensor is disposed on or adjacent to the corresponding distal femur area.

In some examples, the insertion mechanism is configured to impact the broach at a rate of 1 Hz to 100 Hz.

The presently disclosed subject matter also provides a method of impacting a broach/stem into a bone in a proper alignment with real-time feedback. The method includes (a) measuring the differential between multiple sensor signals or a directional alignment sensor signal to determine the alignment of the insertion mechanism; (b) preferably measuring the bone tension via shock waves produced from impacting on a broach/stem and interaction of the shock waves with the bone (i.e., measuring the shock waves produced by the broach/stem being inserted into the bone by an insertion mechanism impacting on a broach/stem handle thereof) or by generating acoustic waves in the broach/stem by a transducer at variable frequency and sensing reflectance of the shock waves from the bone (these signals provide an indication of the bone stress) (c) analyzing the shock waves and/or acoustic waves of the alignment sensor, and preferably also the impact sensor, via an algorithm operated by a processor to derive impact alignment and/or bone stress; and (d) providing feedback to the insertion mechanism to control the force and/or rate of impaction and the alignment of the impaction.

In particular, the presently disclosed subject matter provides a method of inserting a broach, stem, or acetabular cup into a bone using an insertion mechanism, the method including: impacting the broach, stem or acetabular cup into the bone; using the at least one sensor to sense signals (a) produced by the broach or stem or cup during said impacting, or (b) signals generated in the bone as a result of the impacting; (c) analyzing those signals via an algorithm operated by a processor; and (d) providing real-time feedback to control the alignment (and/or bone stress and consequently the force and rate) of impacting.

In some examples, the impaction sensor is an acoustic emission sensor, configured to sense acoustic signals related to the generation of micro-cracks in the bone, and signal sensed by the acoustic emission sensor as well as the other sensors is filtered to differentiate between the impact signal and the signal from the tissue and bone as a precursor to bone fracture.

In some examples, the impacting is performed so that subsequent impacts are within the transient time of a previous impact, thereby resulting in a dynamic coefficient of friction to lower friction between the broach or stem and the bone.

In some examples, the algorithm is configured to control impaction frequency to be within the transient time of each impact.

In some examples, the impacting includes using an inertial insertion mechanism having a lower/impacting mass and an upper mass designed to move in opposite directions to minimize movement of the center of gravity of the insertion mechanism, where only the lower mass operably impacts the broach or stem.

In some examples, the impacting is performed at a rate of 1 Hz to 100 Hz. In some examples, the method involves monitoring the excitation in a longitudinal vibration and/or bending vibration to sense the bone stress.

In other words, the apparatus and method are configured to monitor the broach and stem insertion and or acetabular cup insertion, i.e. monitor the impact alignment and/or bone stress and the corresponding applied force to reduce the risk of bone fracture; and determine the alignment and the impact force (and preferably a movement/displacement force of the broach or stem or acetabular cup). Further, to assure that the alignment is proper, lower force impaction is applied to assure alignment using the at least one sensor that is noncolinear. Then the impaction force is increased in a closed loop using the sensors, to ensure the broach proceeds into the bone while not exceeding a critical level of bone stress as determined by the processor/algorithm. This mitigates the risk of revision surgery, to correct any complications resulting during primary surgery due to fracture, or loosening resulting from inadequate fixation of the stem to the cortex surface of the bone, or incomplete insertion of the acetabular cup. While controlling the force applied, the broach/stem displacement should be large enough to exceed the elastic response of the bone's tissue and assure penetration, i.e. above a “spring-back” elastic bone response.

It is a particular feature of the presently disclosed subject matter that the desired impact on the broach or stem or acetabular cup is determined using shock or acoustic waves as detected by the sensor(s), and shock wave analysis.

Preferably, the impaction at a higher rate than manual impacting/hitting, allowing for impacting at a lower intensity to perform the same penetration rate and reducing risk of bone fracture. Preferably, a subsequent impact is within the transient period that occurs while the broach or stem is still vibrating due to the previous impact, or the shock waves generated from the impact are still not damped, so that the coefficient of friction (COF) between the bone and the implant is dynamic and not static, providing for a more efficient broach insertion. With a high impact rate, the force required is lower as the overall insertion of the broach or stem occurs in multiple small steps.

### BRIEF DESCRIPTION OF DRAWINGS

The presently disclosed subject matter may be more clearly understood upon reading of the following detailed description of non-limiting examples thereof, with reference to the following drawings, in which:

FIG. 1 is a schematic perspective view of an apparatus for inserting broaches and/or a stem in a bone, in accordance with an example of the presently disclosed subject matter.

FIG. 2 is a schematic perspective view of a broach/stem; a broach handle; a sensor; and a sensor holder, in accordance with an example of the presently disclosed subject matter.

FIG. 3 is a perspective view of a portion of an apparatus for providing proper alignment of the broaches and/or stem in a bone, in accordance with an example of the presently disclosed subject matter.

FIG. 4A is a schematic of a tandem piezo transducer arrangement of the insertion mechanism.

FIG. 4B is a graph depicting the resultant beat frequency signal of two piezo transducers in tandem.

FIG. 5 is a side view of the apparatus that replaces a surgical mallet with an insertion mechanism, in accordance with another example of the presently disclosed subject matter.

FIG. 6 is a side view of the apparatus, in accordance with yet another example of the presently disclosed subject matter.

FIGS. 7A and 7B are schematics of an exemplary haptic impact alignment feedback devices for aligning the insertion mechanism of the apparatus of the present apparatus.

FIGS. 8A and 8B are schematics of an exemplary visual impact alignment feedback device for aligning the insertion mechanism of the apparatus of the present apparatus.

FIGS. 9A-9C, are perspective views showing the stem on which a ball joint is mounted (FIGS. 9A and 9B); and acetabular cup that is inserted into the acetabulum prior to ball joint insertion (FIG. 9C).

FIG. 10 is a schematic of a handheld insertion mechanism, in accordance with the present invention.

FIG. 11A is a schematic of a piezo actuator with hydraulic amplification, in accordance with the present invention.

FIG. 11B is a schematic of a piezo impactor with horn amplification, in accordance with the present invention.

The following detailed description of examples of the presently disclosed subject matter refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION

Illustrative examples of a broach/stem insertion/impacting apparatus according to the presently disclosed subject matter are described below. In the interest of simplicity, not all features/components of an actual implementation are necessarily described.

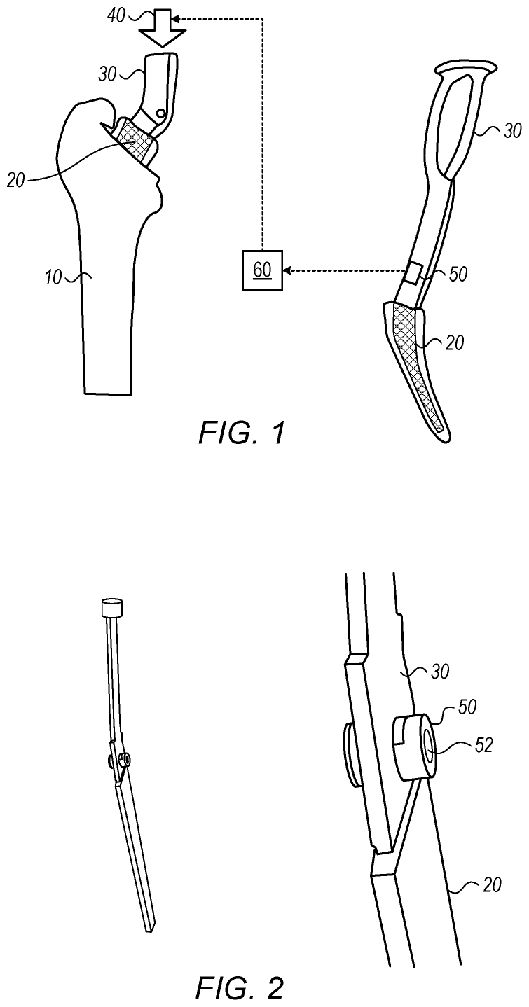

FIG. 1 shows an apparatus for inserting broaches and femoral stems in a bone (e.g. femur 10). The apparatus includes a broach 20 (or femoral stem, in the final insertion—herein after in the specification and claims: “broach”); a broach/stem insertion handle 30; a real time, closed loop insertion mechanism 40 (schematically represented by an arrow); an impaction sensor 50, located at the broach handle; and a processor 60, which are all operably connected to each other. Broach 20 may be a broach as known in the art, to prepare a femoral canal; and so may be broach/stem insertion handle 30. Processor 60 is configured to receive data from one or more sensors 50, analyze that data, whether explicitly or via deep learning, and provide feedback instructions to insertion mechanism 40. In some examples, insertion mechanism 40 has an ergonomic (e.g. elongated) shape so it is easy for hand-held operation by a surgeon, or to be integrated within a robotic platform as part of an impaction end effector.

During a hip replacement procedure, in particular the process of inserting subsequently larger broaches into the femur, each broach 20 is impacted (hit) by insertion mechanism 40. The resulting acoustic vibrations of the impact and its reflection from the bone is sensed by sensor(s) 50. It is also possible to sense the change in the natural frequencies of bone 10 and broach 20 and their damping coefficients, both indicative to the level of fixation of the broach/stem. The aforementioned features of the impact (or the acoustical or vibrational response of the bone due to the impact) are sensed by sensor(s) 50 and conveyed to processor 60, which includes an algorithm for analyzing those particular features with respect to bone stress.

Processor 60 provides results of the algorithm's analysis in order to control insertion mechanism 40, such as the rate and/or force of the impacts. In some examples, sensor(s) 50 is/are located between broach/stem insertion handle 30 and broach 20, or on broach handle 30 as illustrated in FIG. 1; and in other examples, adjacent to the distal end of the femur. FIG. 2, shows sensor 50 held next to broach/stem insertion handle 30, by a sensor holder 52. In other words, there may be more than one sensor 50 in more than one location, as well as more than one type, as understood from the description below. It is noted that the broach/stem area and distal femur area is expected to provide acoustic coupling to the impacts. Processor 60 provides results of the algorithm's analysis in order to control insertion mechanism 40, such as the rate and/or intensity of the impacts.

Sensor 50 (and other sensors noted herein-below) may include at least one of the following: an accelerometer, configured to measure a shock and after shock acceleration; a vibrometer, configured to measure a shock and after shock velocity; a load cell, an interferometer, configured to measure a shock and after shock displacement (amplitude). The vibration has an amplitude (displacement) A; a velocity A*ω, where ω is 2*Pi*F of the vibration frequency F; and an acceleration A*ω\hat{ }2. From this one can understand that for a given A, the position sensor is good at low frequency, the velocity sensor yields a better signal and intermediate frequency and the acceleration will give the strongest signal at high frequency.

Another possible sensor 50 is an acoustic emission sensor, configured to sense acoustic signals related to the generation of micro-cracks in the bone. Micro-cracks can lead to fractures. The signal sensed by sensor 50 (which includes some or all of the sensor types herein) can be processed to differentiate, using time and/or frequency domain separation, between the impact signal and the signal from the tissue and bone, as known per se. In examples where sensor 50 is an acoustic emission device, which senses micro-cracks in brittle materials, the resulting elastic wave frequencies are typically in the tens to hundreds of kHz.

Sensor 50 can optionally be exemplified by one or more of a wide bandwidth microphone (including and exceeding the audible range), configured to measure an acoustic noise and spectrum generated by the impact; and transducers, configured to generate and detect acoustic waves via the broach, stem, or handle, in which the transducer is excited at a desired frequency or frequency range and is coupled to broach 20. The vibrations propagate in broach 20, and waves reflected from the bone, are detected by the transducer. These reflected waves provide information about the bone stress.

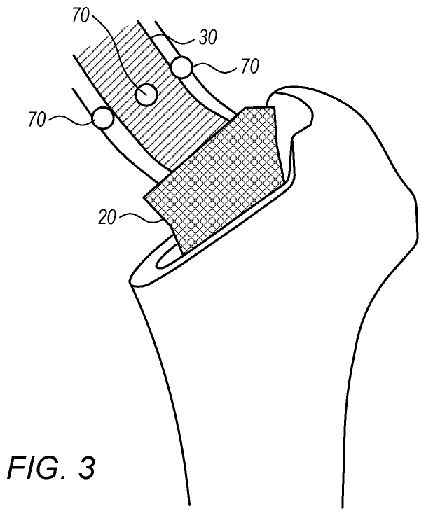

FIG. 3 illustrates a particular feature of the presently disclosed subject matter for ensuring proper alignment of the broaches and/or stem, via feedback from alignment sensors 70 and analysis of the directional force signals by processor 60, for preventing mis-alignment of the impact. These alignment sensors 70 in some examples can also sense impaction, and therefore sometimes be referred to as alignment and impaction sensors, herein-below. At least one direction alignment sensor 70 is located on at least one face of broach/stem insertion handle 30. When multiple alignment sensors 70 are used, they are mounted around broach/stem insertion handle 30. Three alignment sensors 70 are visible. In some examples, four alignment sensors 70 are included. Alignment sensors 70 measure the impaction in several locations around the broach 20 (and stem). The differential signals or the directional force signal are an indication of the impaction direction. In some examples, alignment sensor 70 can be a single directional sensor. In some examples, alignment sensors 70 can also detect bone stress levels.

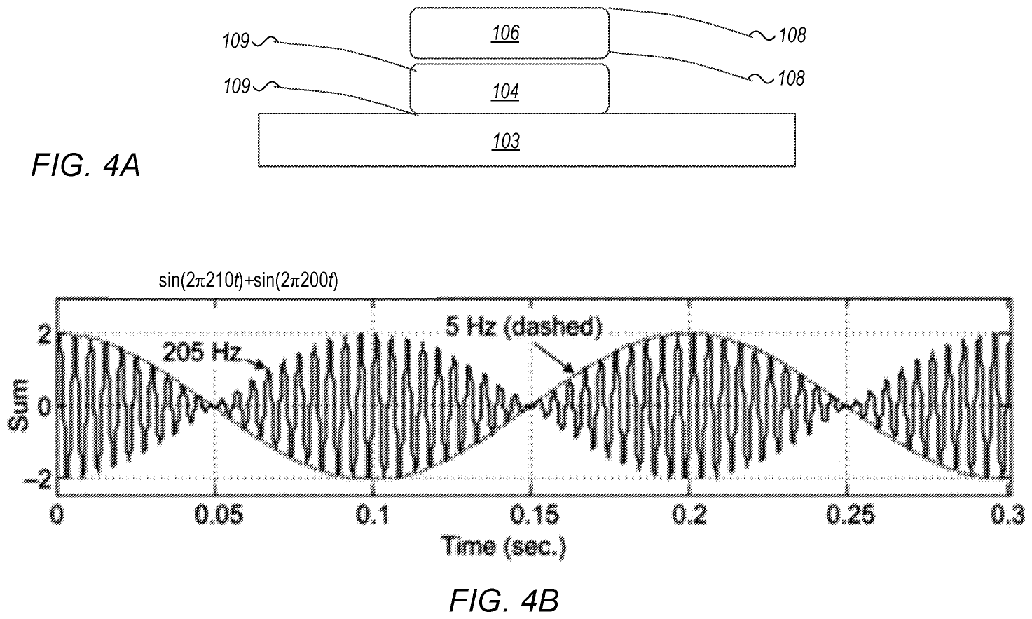

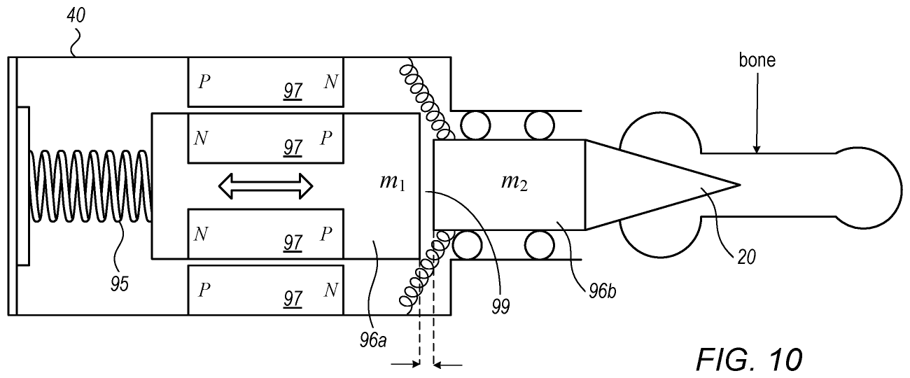

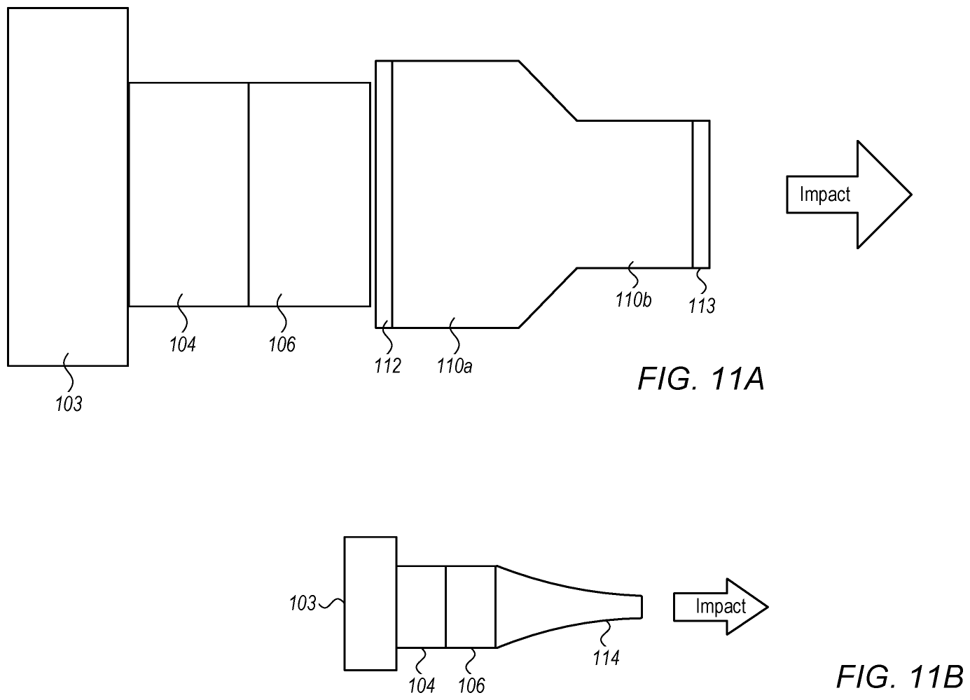

In some examples, insertion mechanism 40 is constituted by an electro-magnetic insertion mechanism (FIG. 10) or a piezo-electric insertion mechanism (FIG. 4A and FIG. 11A).

Using a piezo-electric insertion mechanism (FIG. 4A), a compact, actuator is preferable. It is also preferable to operate the actuator in resonance to maximize impact amplitude. Resonant frequency is inversely proportional to the piezo length. For impaction at 100 Hz, if in resonance, a very long and expensive transducer (actuator) is required. The configuration of the insertion mechanism 40 (FIG. 4A) may include beat frequency generation by two in-resonance piezo-transducers 104 and 106 mounted on a base 103. Piezo-transducers 104 and 106 are driven via wires connection 108 and 109, respectively, to allow for a high amplitude, in resonance, beat signal impacting at variable frequency. The ability to alter the effective impaction frequency facilitates determining and implementing the optimal impaction frequency according to the apparatus algorithm and potentially achieve a minimal insertion friction according to the bone/broach dynamics due to minimization of the coefficient of friction (COF).

The maximum excitation of both piezo transducers 104 and 106 is in the MHz range—a typical resonance range of a thin piezo transducer. Thus the preferable excitation frequency is in that range. Regardless, the frequency shift (difference) between the two MHz driving frequencies could be in the desired 100 Hz range. The following equation describes the summation of two sinusoidal excitations, at frequency f1 and f2:

$$sin ( 2 π {f}_{1} t ) + sin ( 2 π {f}_{2} t ) = 2 cos [ 2 π ( \frac{{f}_{- 1} - {f}_{- 2}}{2} ) t ] \cdot sin [ 2 π ( \frac{{f}_{1} - {f}_{2}}{2} ) t ] . \qquad ( 2 )$$

FIG. 4B shows an example of the resultant signal of the two in-resonance piezo transducers 104 and 106, in tandem. The piezo transducers 104 and 106 are configured to operate at two different frequencies within the resonance range of the piezo transducers, to produce a compact piezo linear actuator, for high resonant signals at low frequency. A resonant frequency is inversely proportional to a piezo transducer's length. The two tandem piezo transducers 104 and 106 in tandem achieve a compact combined transducer yielding sufficient impaction force. The two transducers are activated at different frequencies. The effective response of the two piezo transducer signals in resonance result in a beat frequency of half the sum and half the difference. Half the difference is the desired impaction frequency. Half the sum is a high frequency that is filtered out by the combined broach/stem 20 and bone response time. As a result, in a compact actuator, the impact frequency can be tuned by changing the excitation frequencies of the transducers 104 and 106, while still being in resonance, and therefore achieve the required level of impact force.

In the example of a piezo transducer actuator, there may be either of two additional mechanisms to increase the displacement of insertion mechanism 40. FIG. 11A shows one mechanism embodied by hydraulic pistons 110a, 110b, whereby oscillation amplification of the tandem piezo transducers 104 and 106 is increased by the ratio of input and output piston area. In FIG. 11B, piezo transducers 104 and 106 are attached to a horn 110 with the benefit of travel extension according to the horn geometry selected. Further amplification is possible if the horn resonant frequency is equal to half the difference of the frequencies in FIG. 4A.

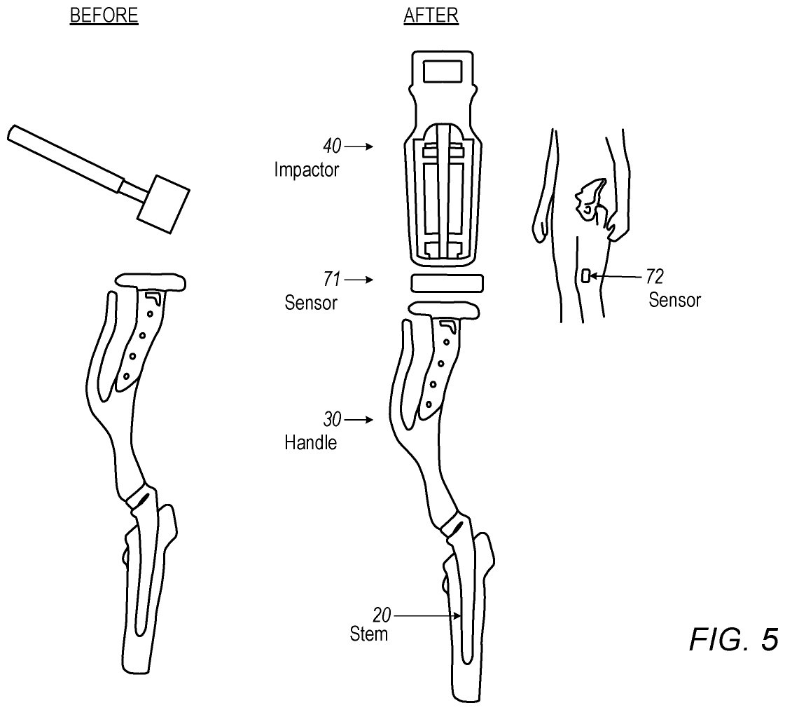

FIG. 5 shows another example of the present apparatus in accordance with the presently disclosed subject matter (labelled “AFTER”), next to a prior art apparatus (labelled “BEFORE”). In this example, alignment and impaction sensor 70 is embodied by a contact plate sensor 71, shown as located below insertion mechanism 40, or a distal femur sensor 72 (although in another example, not shown, contact plate sensor 71 can be located on the broach handle).

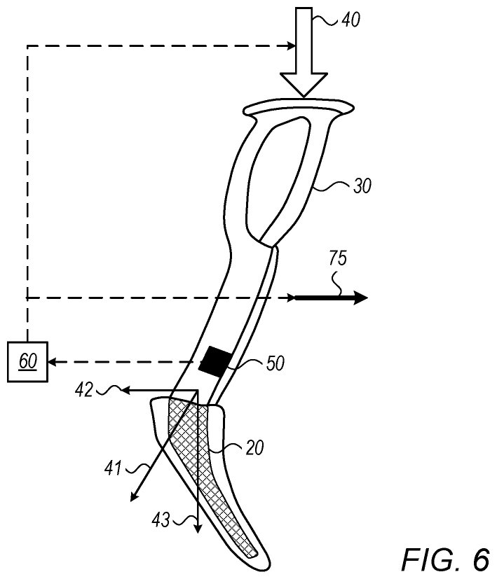

FIG. 6 shows another example of the apparatus including an additional feature to the insertion mechanism, namely, a side impact mechanism 75, which is configured to provide an impact from the side. This side impact may be noncollinear to the main impact direction of insertion mechanism 40, which is generally perpendicular to the main impact, but not necessarily. Side impact mechanism 75 provides a low-force side impact to assure alignment of a force vector 41 of insertion mechanism 40 (broach/stem insertion) along the femur canal, to prevent bone fracture. Force vector 41 is illustrated as separated into a horizontal force vector 42 and a femur canal-aligned force vector 43. Horizontal force vector 42 does not contribute to the broach/stem insertion rather it increases the propensity for bone fracture. An appropriate force applied by side impact mechanism 75 cancels or at least minimizes horizontal force vector 42. Horizontal force vector 42 is determined by sensor 50, which may include a 6DOF accelerometer/gyro. Side impact mechanism 75 is synchronized with the main impact of the insertion mechanism 40 to properly align the force (force vector) of the resultant impact on broach 20. The resultant impaction force vector is thus a combination of the side force vector and the insertion force vector.

By adding a synchronous side impact producible by side impact mechanism 75 of insertion mechanism 40, the resultant direction of the impaction can be optimized to be along the canal of the bone, with a minimal side force. As the impaction is electronically triggered, it is possible to synchronize the two impactions, which is a feature that is impossible with a manual mallet.

With the present apparatus, including side impact mechanism 75, more than one impact can be generated simultaneously so the resultant force is in an optimal direction. Namely, one impact for insertion, and simultaneously also a side impact to minimize stress on the bone during insertion.

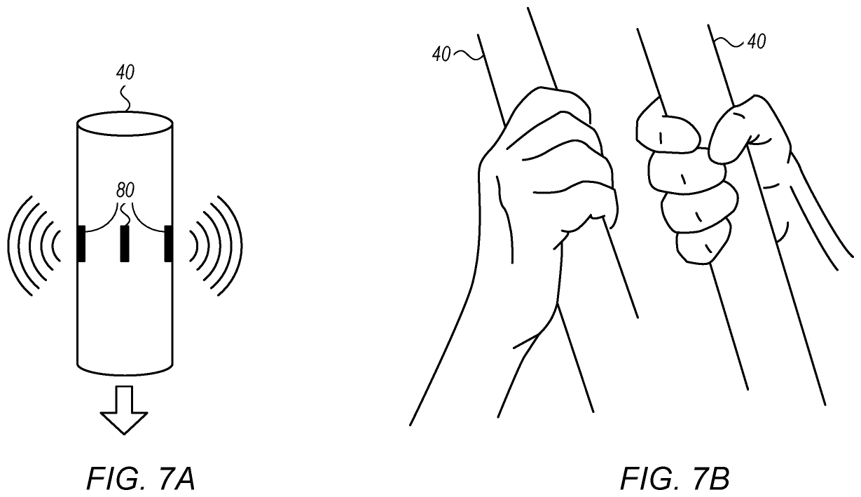

FIGS. 7A and 7B illustrate an exemplary feedback scenario/method to the surgeon using haptic devices associated with insertion mechanism 40 whereby the surgeon receives feedback on how to adjust the angle of the insertion mechanism. For example, there may be four vibrating haptic devices 80 (three visible), whereby the surgeon will align insertion mechanism 40 until a minimal signal difference is achieved in all four directions as sensed by the handheld insertion mechanism 40. The vibrating haptic devices 80 are configured to provide tactile, and/or audible feedback. This alignment process may be executed with a minimal impact, that is an impact sufficient to trigger the alignment sensor(s) 70 to determine (provide feedback to) the alignment. Once aligned, the impact can increase for the actual insertion.

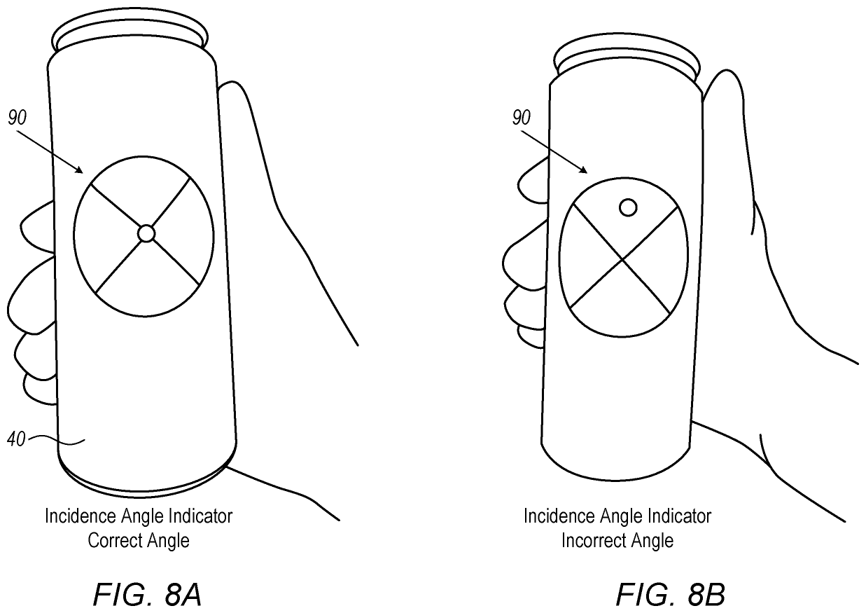

FIGS. 8A and 8B illustrate an exemplary feedback scenario/method using a visual alignment device 90 (depicted as cross-hairs with a target ball or dot) associated with insertion mechanism 40, by which the surgeon receives visual feedback on how to adjust the angle of the insertion mechanism. Processor 60 positions the dot in relation to the cross hairs based on the alignment of insertion mechanism 40 according to corresponding axial and lateral forces. The surgeon aligns insertion mechanism 40 so that the dot or ball is at the intersection of the cross-hairs, preferably during initial, low-force impacts.

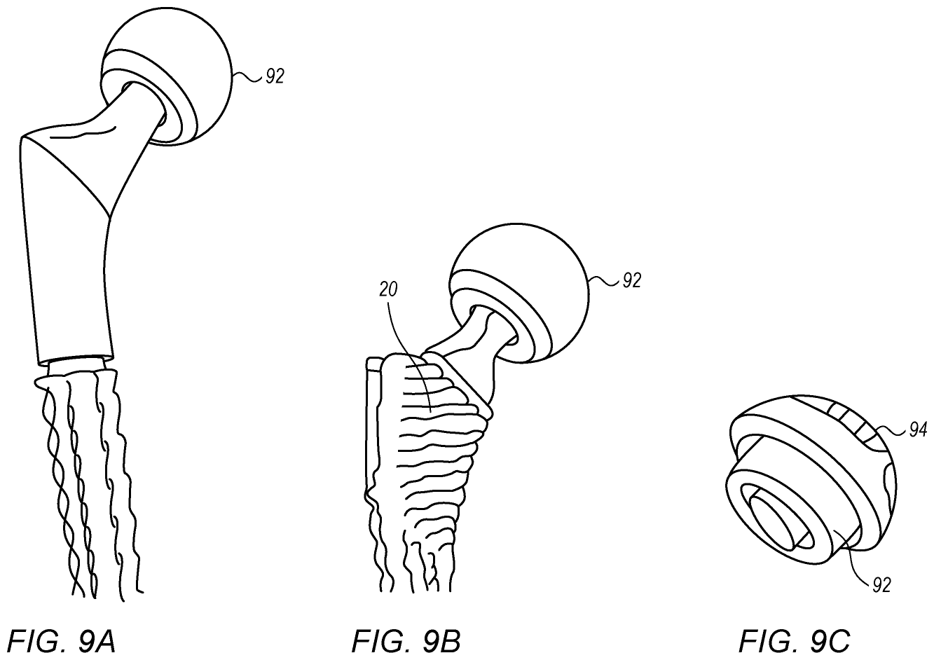

FIGS. 9A and 9B show broach 20, on which an acetabular prosthetic ball joint 92 is mounted to stem 20. FIG. 9C shows an acetabular cup 94, that is inserted into the acetabulum, mounted on the ball joint 92.

FIG. 10 is a schematic diagram showing an insertion device including an electromagnet linear actuator 97; a spring 95; a first movable mass (m1) 96a; and a second movable mass (m2) 96b. Electromagnet linear actuator 97 excites first mass 96a in the resonance frequency of mass 96a and spring 95 to increase the amplitude of vibration. During insertion, first mass (m1) 96a accelerates into a gap 99 and hits the stem 20 via second mass (m2) 96b. The electromagnetic linear actuator 97 of the insertion device is designed to help insert the broach, stem, or cup 94 based on force and alignment feedback to insertion mechanism 40 or to a robotic platform (not shown) used by the surgeon. Second movable mass 96b returns to position via a spring loading.

FIG. 11A shows an example of a mechanical amplifier where the amplitude of oscillation of the piezo transducers 104, 106 is further increased by hydraulic gain using pistons 110a and 110b filled with hydraulic oil and sealed by two movable seals 112 and 113. Upon impaction of the piezo transducers 104, 106, on seal 112, the seal will move thereby driving a larger movement of seal 113, according to the area ratio of the pistons.

FIG. 11B shows another mechanical amplifier that uses a horn 114. An expansion of the piezo transducer will be amplified according to the horn 114 input to output horn area ratio.

A bone density test is a standard measurement deriving various bone parameters commonly measured prior to such medical procedures and can be used as a reference for the algorithm of processor 60 by determining the permitted range of tension according to the bone properties.

Bone Mineral Density (BMD), Body Mass Index (BMI), and a patient's bone density T-score can be included as inputs to the algorithm of processor 60 and tailors the impaction profile of force and frequency to a specific patient. Bone mineral density and the T-score indicate if bones are weaker and thinner than normal and more likely to fracture. BMD is a commonly available test where X-rays can measure how much calcium and minerals are in the femur. Osteoarthritis is often a silent condition, where patients don't feel any symptoms, which is detectable by a bone density test and T-score results, which are usually known prior to a THA procedure.

Obesity, high Body Mass Index (BMI), affects bone quality in complex ways and increases the risk of fractures for a given bone mineral density (BMD) in obese patients. High BMI adversely affects bone health by alteration of bone-regulating hormones; increased oxidative stress; joint inflammation; and altered bone cell metabolism. BMI can also be an input to the algorithm of processor 60.

In some examples, an AI system is incorporated in processor 60 to determine when critical bone stress is being approached by the impacting.

Sensors 50, 70, 71, and 72 each sense impact and provide raw data for an early detection of approaching the optimal insertion, and control the impact frequency and intensity (force)

It should be understood that the above description is merely exemplary and various examples of the present presently disclosed subject matter may be devised, mutatis mutandis, and that the features described in the above-described examples, and those not described herein, may be used separately or in any suitable combination; and the presently disclosed subject matter can be devised in accordance with examples not necessarily described above.

1. An apparatus for inserting a femoral broach or femoral stem or acetabular cup into a bone, the broach, the stem, and the cup having an associated handle for impaction and insertion, the apparatus comprising:
at least one closed loop insertion mechanism configured to provide impacts on the associated handles of the femoral broach, or the femoral stem, or the acetabular cup;
at least one sensor configured to sense a series of impacts, including impact angle, and to provide real-time feedback of the alignment and/or condition of the bone; and
a processor operably connected to the at least one sensor, the processor comprises an algorithm configured to receive the real-time feedback from the at least one sensor, and operably connected to the insertion mechanism to control the force, frequency, and alignment of the impacts by the at least one closed loop insertion mechanism.

2. The apparatus of claim 1, wherein the at least one sensor is any one or combination of an accelerometer, a vibrometer, a load cell, an interferometer or position sensor, a wide band microphone, an acoustic emission sensor, and a transducer.

3. The apparatus of claim 1, wherein the at least one sensor is configured to provide real-time feedback based on shock-induced vibrational data or acoustic data.

4. The apparatus of claim 1, wherein the at least one sensor is disposed on at least one face of the broach, the stem, or the cup handles.

5. The apparatus of claim 1, wherein the at least one sensor detects impact alignment and/or bone stress level.

6. The apparatus of claim 1, wherein the at least one closed loop insertion mechanism comprises a side impact mechanism.

7. The apparatus of claim 1, wherein the processor comprises a force analysis algorithm and an alignment analysis algorithm to control or stop insertion according to the bone condition.

8. The apparatus of claim 7, wherein the force analysis algorithm is configured to control or stop insertion is based on preoperative information of each patient.

9. The apparatus of claim 1, wherein the processor is configured to adjust the angle of the insertion mechanism in accordance with feedback from the at least one sensor.

10. The apparatus of claim 1, wherein the at least one sensor is activated by a minimal impaction force to provide real-time feedback for the alignment of the insertion mechanism.

11. The apparatus of claim 1, wherein the at least one sensor is mounted on a distal end of a femur to detect bone condition changes and potential bone fracture.

12. The apparatus of claim 1, configured to predict and avoid intraoperative periprosthetic femoral fractures in real-time by using bone condition feedback signals during the insertion of the broach and/or the stem into the femur.

13. The apparatus of claim 1, wherein at least one closed loop insertion mechanism is configured to be handheld or integrated within a robotic platform, both being controlled by alignment, frequency, and force feedback from the at least one sensor and algorithm.

14. The apparatus of claim 1, wherein it is configured to be handheld and to provide real time visual, and/or tactile, and/or audible feedback to a surgeon about patient bone condition and broach and/or stem, and/or cup insertion status.

15. The apparatus of claim 1, wherein it is configured to be integrated with a robotic platform to indicate real time visual, and/or tactile, and/or audible feedback to a surgeon about patient bone condition and broach and/or stem, and/or cup, insertion will be indicated by the robotic platform user interface.

16. The apparatus of claim 1, wherein the at least one sensor, comprises an acoustic emission sensor, which is configured to sense acoustic signals related to the generation of micro-cracks in the bone, and the signals sensed by the acoustic emission sensor are processed by the algorithm to indicate bone condition changes and when a fracture is about to occur.

17. The apparatus of claim 1, wherein the algorithm is configured to control impact frequency so that subsequent impacts occur within the transient time of the previous impact, thereby reducing dynamic coefficient of friction to help safe insertion of the broach/stem.

18. The apparatus of claim 1, wherein the insertion mechanism is controlled based on a feedback signal from the at least one sensor to indicate when full insertion of the cup within the acetabulum has been reached.

19. A method of inserting a broach, a stem, or an acetabular cup into a bone using an insertion mechanism, the method comprising:
impacting the broach, the stem, or the acetabular cup for insertion into the bone;
using at least one sensor to sense signals (a) resulting from the broach, stem, or acetabular cup insertion, or (b) generated in the bone as a result of the impacting;
analyzing said signals via an algorithm operated by a processor; and
providing real-time feedback to a surgeon to control the force and angle of impacting.

20. The method of claim 19, further comprising sensing acoustic signals related to potential micro-cracks and/or potential fractures in the bone; and
differentiating between the impact signal and the signal from the bone, sensed by an acoustic emission sensor.

21. The method of claim 19, further comprising using a Fast Fourier Transform (FFT) to determine stable fixation of the broach, the stem, or the acetabular cup into the bone.

22. The method of claim 19, further comprising performing a frequency sweep by a first transducer; and measuring a frequency response of the bone by a second transducer, to help determine fixation stability of the broach, stem, or cup.

23. An electromagnetic insertion mechanism for inserting a femoral broach, or a femoral stem, or an acetabular cup into a bone, the mechanism comprising:
an electromagnetic or piezo linear actuator,
a first movable mass,
a second movable mass, and
a spring, in resonance with the first mass,
the electromagnetic or piezo linear actuator configured to excite the first movable mass, which hits the second movable mass to thereby impact the broach, the stem, or the cup.

24. The insertion mechanism of claim 23, wherein it is configured to be handheld and to provide real time visual, and/or tactile, and/or audible feedback to a surgeon about patient bone condition and broach and/or stem, and/or cup insertion status.

25. The insertion mechanism of claim 23, wherein the mechanical response of the piezo linear actuator is enhanced by hydraulic gain mechanism or a horn.

26. The insertion mechanism of claim 23, further comprising a synchronous noncollinear side impaction mechanism to minimize side forces of the broach or stem on the bone.

27. The insertion mechanism of claim 23, is configured to be handheld or integrated within a robotic platform.

28. The insertion mechanism of claim 23, wherein it is configured to be integrated with a robotic platform to indicate real time visual, and/or tactile, and/or audible feedback to a surgeon about patient bone condition and broach and/or stem, and/or cup, insertion will be indicated by the robotic platform user interface

29. The insertion mechanism of claim 23, wherein the two piezo actuators are arranged in in tandem.

30. The insertion mechanism of claim 29, wherein the piezo linear actuator comprises two in-resonance piezo transducers in tandem, which are configured to operate at two different frequencies within the resonance range of the transducers.
